(19) United States
(12) Patent Application Publication
(10) Pub. No.: US 2009/0171370 A1
(43) Pub. Date: Jul. 2, 2009

(54) Navigation System for Hip Replacement Surgery Having Reference Mechanism and Method Using the Same

(75) Inventors: Yong San Yoon, Daejon (KR); Young Yong Kim, Yongin-si (KR); Jerome Alain Tonetti, Grenoble (FR)

Correspondence Address: BACON & THOMAS, PLLC, 625 SLATERS LANE, FOURTH FLOOR, ALEXANDRIA, VA, 22314-1176, US

(73) Assignee: Korea Advanced Institute of Science and Technology, Daejon (KR)

(21) Appl. No.: 11/918,342

(22) PCT Filed: Apr. 12, 2006
(86) PCT No.: PCT/KR2006/001335

§ 371 Date: Jan. 14, 2009

(30) Foreign Application Priority Data
Apr. 12, 2005 (KR) 10-2005-0030405

(51) Int. Cl. A61B 19/00

(52) U.S. Cl. 606/130

(57) ABSTRACT

A navigation system for an acetabular cup, which guides an insertion orientation of the acetabular cup inserted into a pelvis during a total hip replacement surgery, includes: a pelvis position tracer which includes probes in contact with three particular points of the pelvis placed on an anterior pelvic plane and a first reference mechanism disposed to indicate a specific reference plane when the probes come in contact with the particular points; and a pelvis position indicator which is fixed to the pelvis, and includes a second reference mechanism that is adjustable to indicate a plane parallel to the specific reference plane indicated by the first reference mechanism, or to indicate a plane perpendicular thereto, or to indicate the both planes. Accordingly, an insertion orientation of an acetabular cup can be guided by using a reference mechanism having a simple structure, and the acetabular cup can be accurately guided regardless of changes in the patient's pelvic position during surgery, because a plane used in the insertion of the acetabular cup can be indicated continuously.

### TECHNICAL FIELD

The present invention relates to a navigation system for an acetabular cup, and more particularly, to a navigation system for an acetabular cup, which guides insertion orientation of the acetabular cup in hip replacement surgery by using a reference mechanism used for indicating a plane parallel to an anterior pelvic plane or a plane tilted at a specific angle.

### BACKGROUND ART

The hip joint is located between the ball-shaped femoral head of the femur and the socket-shaped acetabulum of the pelvic bone wrapping around the femoral head. When the hip joint is severely damaged for various reasons, an artificial hip joint surgery is performed by removing a joint portion. This is called a total hip replacement or a hip arthroplasty. An artificial hip joint is composed of a portion inserted into the femur to substitute for the femoral head and a portion inserted into the pelvis to substitute for the acetabulum.

In the total hip replacement, the acetabular cup substituting for the acetabulum has to be inserted into the pelvis in a correct orientation. An insertion orientation of the acetabular cup is determined based on an anterior pelvic plane that is defined by three points in the pelvis, that is, a left anterior superior iliac spine, a right anterior superior iliac spine, and a symphysis pubis. Incorrect insertion of the acetabular cup may shorten the lifespan of the artificial hip joint, and even may cause dislocation.

To avoid this, a navigation system for the acetabular cup has been proposed, which accurately guides the insertion orientation of the acetabular cup.

An optical navigation system for the acetabular cup is disclosed in U.S. Pat. No. 5,141,512. The system includes a light source having an angle adjusting element, and three foot portions. The foot portions are respectively fixed to the aforementioned three points of the pelvis. The angle adjusting element controls a direction of a light beam emitted from the light source, so that the direction corresponds to the insertion orientation of the acetabular cup. The light beam is reflected from a mirror mounted on an acetabular cup inserter. When incident and reflected beams are coincident, the acetabular cup is aligned for correct placement. However, this system is unable to accommodate variation in the patient's pelvic position during surgery, since the foot portions are fixed to the system. In addition, the light beam is blocked by other surgical equipments, encumbering an alignment operation.

A computer assisted navigation system for a hip replacement surgery is disclosed in U.S. Pat. No. 6,711,431. The system defines a patient's pelvic plane with reference to at least three pelvic points, and traces a pelvic tracking marker, fixable to the pelvic bone, by using a location tracking device, thereby tracking in real time the orientation of the defined pelvic plane. The system can trace the patient's pelvic plane regardless of variation in the patient's pelvic position. However, infection may occur due to wire cables, and the surgery may be encumbered by the magnitude of the system when performed in a narrow operating room. In addition, the system is relatively expensive.

### DETAILED DESCRIPTION OF THE INVENTION

### Technical Goal of the Invention

In order to solve the aforementioned problems, an object of the present invention is to provide a navigation system for an acetabular cup, which guides an insertion orientation of the acetabular cup by using a reference mechanism having a simple structure, without the use of an electric device, and a method thereof.

Another object of the present invention is to provide a navigation system for an acetabular cup, which includes a reference mechanism capable of indicating a plane continuously, regardless of variation in the patient's pelvic position during surgery, where the plane is referenced in the insertion of the acetabular cup, and a method thereof.

### DISCLOSURE OF THE INVENTION

According to an aspect of the present invention, there is provided a navigation system for an acetabular cup, which guides an insertion orientation of the acetabular cup inserted into a pelvis during a total hip replacement surgery, and uses reference mechanisms, the navigation system comprising: a pelvis position tracer which includes probes in contact with three particular points of the pelvis placed on an anterior pelvic plane and a first reference mechanism disposed to indicate a specific reference plane when the probes come in contact with the particular points; and a pelvis position indicator which is fixed to the pelvis, and includes a second reference mechanism that is adjustable to indicate a plane parallel to the specific reference plane indicated by the first reference mechanism, or to indicate a plane perpendicular thereto, or to indicate the both planes.

According to another aspect of the present invention, there is provided a method of guiding an insertion orientation of an acetabular cup inserted into a pelvis during a total hip replacement surgery by using a navigation system for the acetabular cup, where the navigation system uses the reference mechanisms of claim 1, the method comprising: (a) fixing the pelvis position indicator to the pelvis; (b) allowing the first reference mechanism to indicate a specific reference plane by arranging the pelvis position tracer so that the probes of the pelvis position tracer come in contact with the three particular points; (c) fixing the second reference mechanism of the pelvis position indicator after adjusting the second reference mechanism to indicate a plane parallel to the specific reference plane indicated by the first reference mechanism of the pelvis position tracer and/or a plane perpendicular the plane parallel to the specific reference plane; (d) removing the pelvis position tracer arranged on the pelvis; (e) separating the pelvis position indicator from the pelvis; (f) disposing the pelvis to a desirable position; (g) fixing again the pelvis position indicator to the position where the pelvis position indicator is fixed in the (a); and (h) navigating the acetabular cup with reference to the plane indicated by the second reference mechanism of the pelvis position indicator.

### BRIEF DESCRIPTION OF THE DRAWINGS

FIG. 1 is a perspective view of a pelvis position tracer according to an embodiment of the present invention;

FIG. 2 is an exploded perspective view of a pelvis position indicator according to an embodiment of the present invention;

FIG. 3 is a cross-sectional view of the assembled pelvis position indicator of FIG. 2;

FIG. 4 is a perspective view of a typical acetabular cup;

FIG. 5 is a front view of a pelvis, illustrating an anterior pelvic plane;

FIG. 6 is a side view of the pelvis of FIG. 5;

FIG. 7 shows locations of the devices of FIGS. 1 and 2 in use;

FIG. 8 is a perspective view of a pelvis position indicator according to another embodiment of the present invention;

FIG. 9 is a perspective view of a pelvis position tracer according to another embodiment of the present invention;

FIG. 10 is a perspective view of a pelvis position tracer having a laser level tool according to another embodiment of the present invention; and

FIG. 11 shows the pelvis position tracer of FIG. 10 in use, illustrating a corresponding pelvis position indicator.

### BEST MODE FOR CARRYING OUT THE INVENTION

The attached drawings for illustrating exemplary embodiments of the present invention are referred to in order to describe clearly the aforementioned features or other features of the present invention.

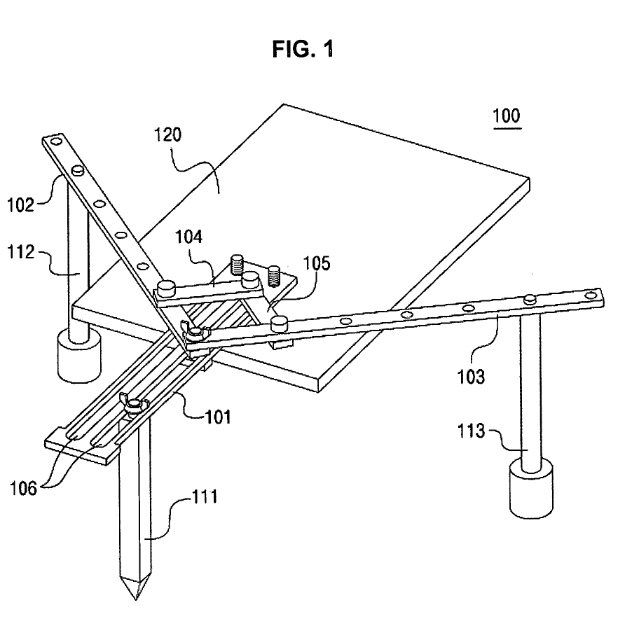

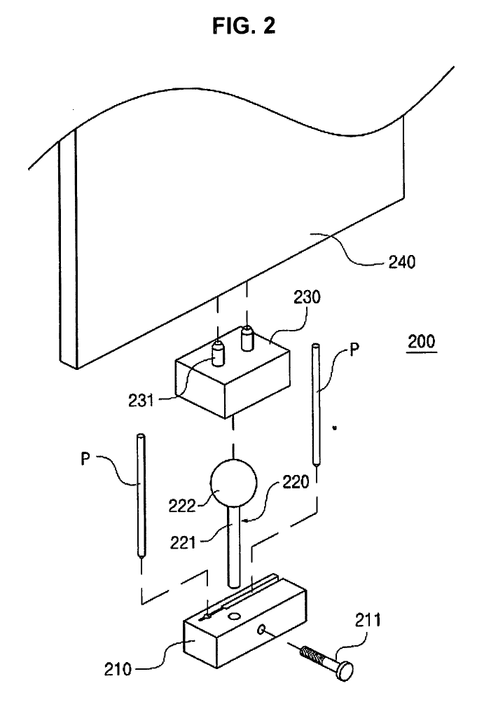

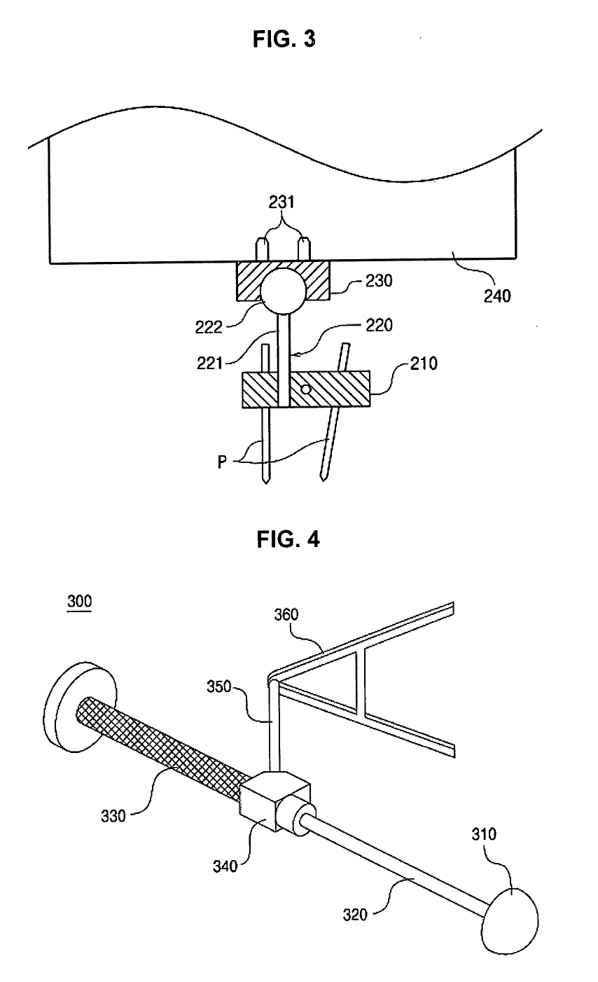

FIG. 1 is a perspective view of a pelvis position tracer according to an embodiment of the present invention. FIG. 2 is an exploded perspective view of a pelvis position indicator according to an embodiment of the present invention. FIG. 3 is a cross-sectional view of the pelvis position indicator of FIG. 2.

As shown in FIG. 1, in a pelvis position tracer 100, three members 101, 102, and 103 are joined in the form of Y generally, probes 111, 112, and 113 are included in the members 101, 102, and 103, and a reference mechanism 120 is fixed to a first end of the first member 101. A lengthwise hole 106 is provided at the first member 101 which is disposed at the center of the device 100 to support other members 102 and 103. The second member 102 at the left and the third member 103 at the right are connected in such a way that respective first ends thereof are rotatably connected with each other by means of a connecting pin. The connecting pin is inserted into the lengthwise hole 106 of the first member 101. The connecting pin can move along the lengthwise hole 106 of the first member 101. Two link members 104 and 105 which are shorter than the second and third members 102 and 103 are respectively connected to the second and third members 102 and 103. Also, respective first ends of the link members 104 and 105 are rotatably connected by means of a pin or its equivalent, and are then fixed to the first member 101. By doing so, when the connecting pin is moved along the lengthwise hole 106 of the first member 101, the second and third members 102 and 103 may narrow or extend their gaps in a link manner. Preferably, the second and third members 102 and 103 do not move after they are suitably disposed. Therefore, the connecting pin may be fixed by means of a screw or its equivalent. One or more lengthwise holes 106 may be provided.

Further, in the lengthwise hole 106 of the first member 101, the probe 111 can move along the longitudinal direction of the hole 106. Preferably, the probe 111 is so constructed that a user can fix the probe 111 to a desirable position by using a screw or its equivalent. Also, the second and third members 102 and 103 are respectively connected to the probes 112 and 113. As shown in FIG. 1, a plurality of holes used for fixing the probes 112 and 113 to a desirable position may be provided at the members 102 and 103. Each probe comes in contact with particular points of the pelvis, and thereafter the user can trace an anterior pelvic plane. For example, the user may bring probe 111 fixed to the first member 101 into contact with the symphysis pubis, and bring the probes 112 and 113 fixed to the second and third members 102 and 103 into contact with the left and right anterior superior iliac spines.

A first end of the probe 111, in contact with the symphysis pubis, may be curved in the L shape as shown in FIG. 9. If the pelvis is narrow, a screw for fixing the second and third member 102 and 103 and a screw for fixing the probe 111 may interfere with each other. However, if the first end of the probe 111 is curved, and the curved portion is brought into contact with the symphysis pubis, the screw for fixing the probe 111 can move downwards by as much as the length of the curved portion. Accordingly, the screw for fixing the probe 111 and the screw for fixing the second and third member 102 and 103 do not interfere with each other.

The flat plane-shaped reference mechanism 120 is disposed at a first end of the first member 101. As mentioned above, when the probes 111, 112, and 113 come in contact with three points in the pelvis, that is, the symphysis pubis, the left anterior superior iliac spine, and the right anterior superior iliac spine, the reference mechanism 120 indicates a plane parallel to the anterior pelvic plane. In this embodiment of the present invention, the flat reference mechanism 120 is used, but different reference mechanisms of various forms may be used. For example, two rod-shaped members may be used, where one is disposed to indicate the vertical axis of the anterior pelvic plane, and the other is disposed to indicate the horizontal axis of the anterior pelvic plane. In addition, although the reference mechanism 120 is disposed to indicate the plane parallel to the anterior pelvic plane in this embodiment, the present invention is not limited thereto, and the reference mechanism 120 may be parallel to the sagittal plane, that is, the anterior pelvic plane.

As shown in FIGS. 2 and 3, the pelvis position indicator includes a supporting block 210 which is fixed to the pelvis by means of a fixing element such as one or more pins P, a ball joint 220 fixed to the supporting block 210, a fixed block 230 connected to the ball joint 220, and a reference mechanism 240 placed at the fixed block 230.

The supporting block 210 is fixed to the pelvis by means of the pin P, and supports other members. Two pins P are used here, but the present invention is not limited thereto, and one pin P, or three or more pins P may be used. A slot piercing through the supporting block 210 in the longitudinal direction is provided, and a slot space is regulated by a screw 211. The pin P fixed to the pelvis is inserted to the slot of the supporting block 210, and the screw 211 is tightened, thereby fixing the supporting block 210. The supporting block 210 has a hole through which the ball joint 220 is inserted.

The ball joint 220 includes a sphere head 222 and a foot portion 211 which extends from the sphere head 222 and is inserted into the hole included in the supporting block 210. The sphere head 222 of the ball joint 220 is connected to the fixed block 230.

The fixed block 230 has its interior connected to the sphere head 222 of the ball joint 220. In a room temperature, the interior of the fixed block 230 may shrink to tighten the sphere head 222 of the ball joint 220 firmly. In a specific temperature range, the interior of the fixed block 230 may not tighten the sphere head 222 of the ball joint 220 firmly, and thus the fixed block 230 may rotate about the ball joint 220. For this, the fixed block 230 may be made of a shape memory alloy. Preferably, the fixed block 230 is not separated from the ball joint 220, even though it may rotate about the ball joint 220 in a specific temperature range. For convenience, the specific temperature range may be a sterilizing temperature for surgical equipments.

The reference mechanism 240 is flat plane-shaped, and is connected to the fixed block 230 by means of two protrusions 231 included in the fixed block 230. For this, the reference mechanism 240 has two holes through which the protrusions 231 are inserted. Similarly to the pelvis position tracer 100, the reference mechanism 240 may have a different shape besides the flat plane shape.

FIG. 4 is a perspective view of a commercially available acetabular cup, according to an embodiment of the present invention.

An acetabular cup inserter 300 includes a pole-shaped member 320, whose front end is fixed to an acetabular cup 310, a grip 330, and a reference mechanism 360 used in checking an insertion angle. In the device of FIG. 4, a supporting rod 350 extends from a block 340 disposed between the pole-shaped member 320 and the grip 330 at a specific angle with respect to the longitudinal direction of the pole-shaped member 320. In addition, the A-shaped reference mechanism 360 is fixed to an end of the supporting rod 350.

Hereinafter, a method of guiding an insertion orientation of the acetabular cup inserter 300 by using the aforementioned pelvis position tracer 100 and pelvis position indicator 200 will be described.

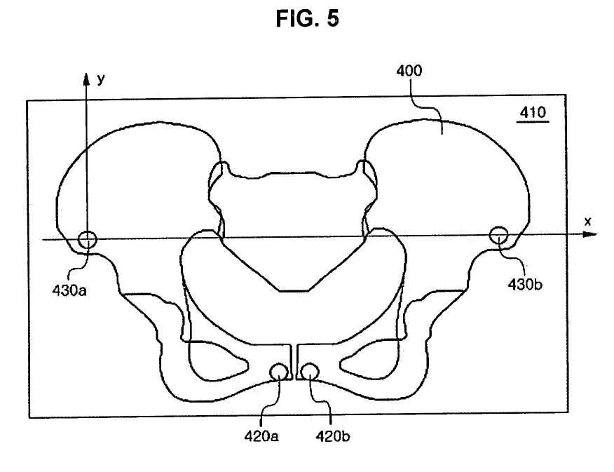

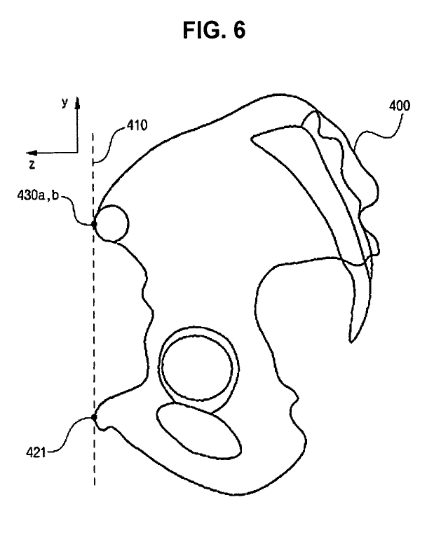

First, the user respectively brings the three probes 111, 112, and 113 of the pelvis position tracer 100 into contact with the three points in the pelvis, that is, the symphysis pubis, the left anterior superior iliac spine, and the right anterior superior iliac spine. The three points are shown in FIGS. 5 and 6, which are a front view of a pelvis 400, and a side view of the pelvis 400, respectively. In FIGS. 5 and 6, the symphysis pubis is designated by reference 420a or 420b, and the left and right anterior superior iliac spines are designated by references 430a and 430b, respectively. An anterior pelvic plane 410 means a plane including the three points.

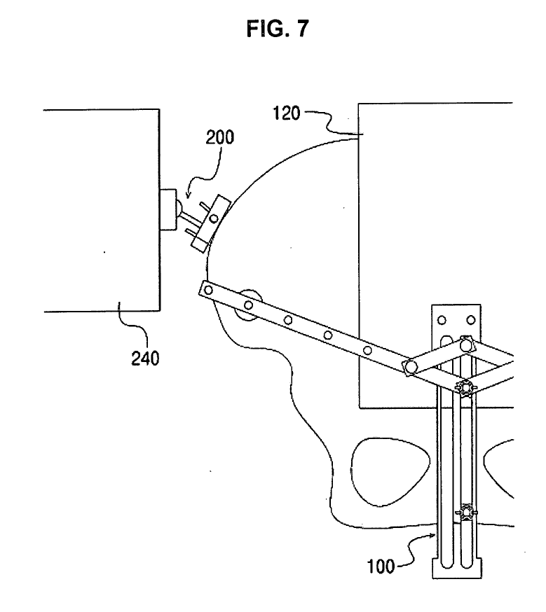

When the probes of the pelvis position tracer 100 comes in contact with the three points respectively, the reference mechanism 120 of the pelvis position tracer 100 is disposed parallel to the anterior pelvic plane 410. This is shown in FIG. 7.

Next, the pelvis position indicator 200 is fixed to the pelvis by means of a pin that is pre-fixed to one point of the pelvis. In FIG. 7, the pelvis position indicator 200 is fixed near the left anterior superior iliac spine. Here, the fixed block 230 is in a specific temperature range by heating. In this condition, the user may dispose the reference mechanism 240 of the pelvis position indicator 200 by regulating the fixed block 230, so that the reference mechanism 240 indicates a plane which is parallel to the plane indicated by the reference mechanism 120 of the pelvis position tracer 100. When the temperature of the fixed block 230 drops to the room temperature, and is then fixed to the ball joint 220 firmly, the user removes the pelvis position tracer 100 placed on the pelvis. Then, the user separates the fixed block 210 from the pin P by regulating the fixed screw 211 included in the supporting block 210 of the pelvis position indicator 200. Now, since the pin P only is fixed to the pelvis, the patient's pelvic position can change easily to a desirable position.

Again, the pelvis position indicator 200 is fixed to the pelvis, after changing the pelvic position to the desirable position. Since the fixed block 230 and the ball joint 220 are firmly bonded, the reference mechanism 240 still indicates the plane parallel to the anterior pelvic plane.

With reference to the reference mechanism 240 of the pelvis position indicator 200, the user navigates the acetabular cup inserter 300, and then inserts the acetabular cup into a desirable position.

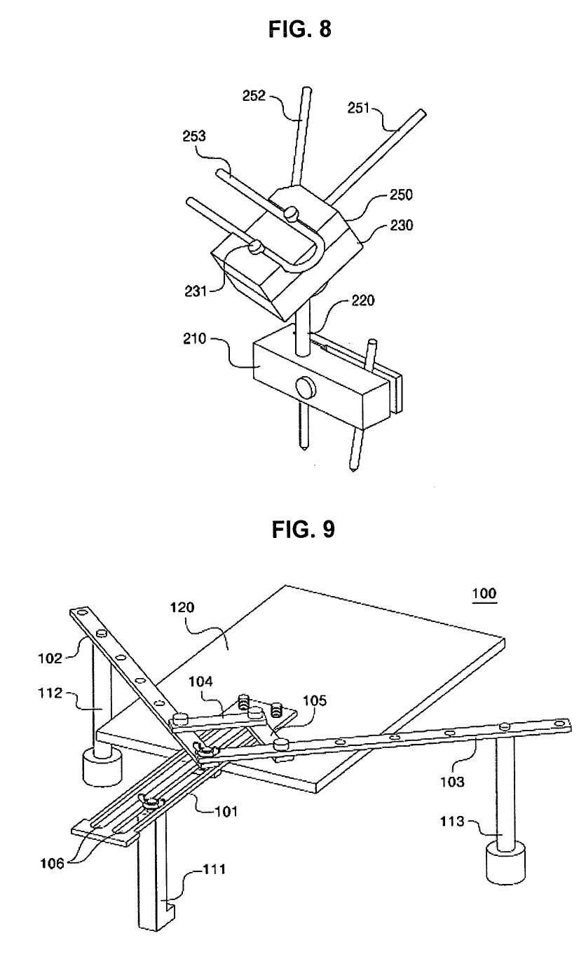

FIG. 8 is a perspective view of the pelvis position indicator 200 according to another embodiment of the present invention. The device has the same configuration of that described in the previous embodiment of the present invention, except for the shape of the reference mechanism. In FIG. 8, therefore, like numeral references with respect to FIG. 2 denote like elements.

The pelvis position indicator of FIG. 8 includes two rod-shaped members 251 and 252 as reference mechanisms. The rod-shaped members 251 and 252 are inserted into a block 250, and the block 250 is fixed to the fixed block 230 in a detachable manner. The block 250 has a hole through which protrusions 231 of the fixed block 230 is pierced and inserted. The block 250 has a slot in its center, and a fixed pin 253 is inserted into the slot. The fixed pin 253 is engaged with notches included in the protrusion 231, and prevents the block 250 from separating from the fixed block 230. A tilted plane is formed at one edge of the block 250, and a hole through which any rod-shaped reference mechanism is inserted is disposed in the tilted plane. Then, the two reference mechanisms can be directed in different directions with each other. In FIG. 8, the rod-shaped reference mechanisms are disposed to indicate a plane perpendicular to the anterior pelvic plane.

The user can use a single type reference mechanism only, or can use two types of reference mechanisms, if necessary. For example, the user may dispose the pelvis position indicator 200, so that it indicates the anterior pelvic plane by using the flat plane-shaped reference mechanism, and then guide the acetabular cup inserter 300 according to the result thereof. Thereafter, the user may remove the flat-shaped reference mechanism, then place the rod-shaped reference mechanism, and then guide the acetabular cup inserter additionally.

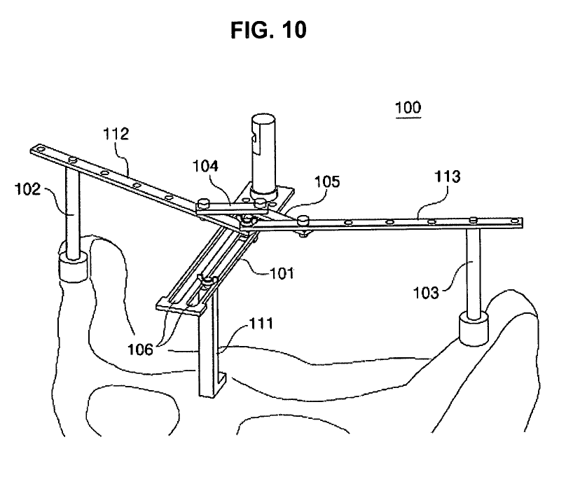

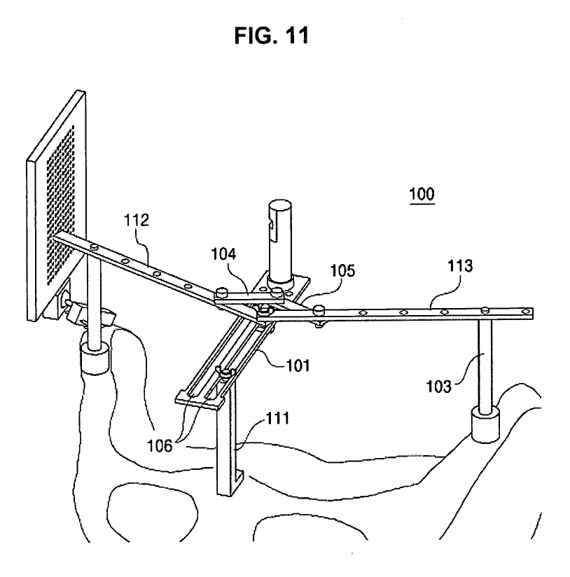

FIGS. 10 and 11 are views of the pelvis position tracer 100 and the pelvis position indicator 200 according to another embodiment of the present invention. Similarly, like numeral references with respect to FIG. 2 denote like elements.

The pelvis position tracer 100 of this embodiment indicates a reference plane by using a laser beam. The pelvis position tracer 100 includes a laser level tool 150 which emits the laser beam used in indicating a specific plane. The laser level tool 150 is commercially available, and may indicate a horizontal plane, that is, a plane parallel to the surface on which the laser level tool 150 is placed. Further, the laser level tool 150 may indicate not only the horizontal plane but also a vertical plane and a plane at a user-defined height. In this embodiment, it is sufficient if the laser level tool 150 is able to indicate the anterior pelvic plane and/or the sagittal plane. For example, the laser level tool 150 may emit a laser beam for indicating a plane parallel to the anterior pelvic plane. The laser level tool 150 may be disposed at any position where the laser beam can be emitted without interference with other members.

With reference to the laser beam emitted from the laser level tool 150, the pelvis position indicator 200 of the present embodiment includes a second reference mechanism 240a which indicates a plane perpendicular to the plane indicated by the laser beam. The second reference mechanism 240a is flat polygonal plane-shaped, and, particularly in this embodiment, is rectangular flat plane-shaped as shown in FIG. 11. The flat plane has a plurality of through-holes along the rectangular surface. For example, 100 through-holes may be horizontally provided in the plane, and 100 through-holes may be vertically provided in the plane.

A method of guiding the acetabular cup by using the pelvis position tracer 100 and the pelvis position indicator 200, according to this embodiment of the present invention will now be described.

When the pelvis position tracer comes in contact with the three particular points of the pelvis, the laser level tool 150 placed in the pelvis position tracer emits a laser beam indicating the plane parallel to the anterior pelvic plane. Then, by adjusting the second reference mechanism 240a included in the pelvis position indicate device, the laser beam emitted from the laser level tool 150 passes through the through-holes of the second reference mechanism 240a. Since the second reference mechanism 240a is flat plane-shaped, and has a specific thickness, if it is not perpendicular to the laser beam, the laser beam is blocked, and thus fails to pass through the through-holes. For this reason, if the second reference mechanism 240a is disposed such that the laser beam can pass through the through-holes of the second reference mechanism 240a, the second reference mechanism 240a indicates a plane perpendicular to the plane indicated by the laser beam. After the second reference mechanism 240a is adjusted, and its position is then fixed, the pelvis position tracer and the pelvis position indicator are removed from the pelvis. Then, the user can easily place the pelvis to a position suitable for surgery. Again, the pelvis position indicator is fixed, after the placement of the pelvis, and then the second reference mechanism 240a guides the navigation of the acetabular cup in reference to the plane indicated by the second reference mechanism 240a.

In another embodiment of the present invention, the second reference mechanism 240a may be a flat plane-shaped member having a mirror surface, instead of having a plurality of through-holes. In this case, a first reference mechanism includes a laser level tool, which emits a laser beam indicating a specific reference plane, and a rod-shaped reference mechanism indicating a direction perpendicular to the specific reference plane. The second reference mechanism includes a reference mechanism, which has a mirror surface to reflect a laser beam emitted from the laser level tool of the first reference mechanism, and a rod-shaped reference mechanism which is fixed to the reference mechanism having the mirror surface, and is parallel to the mirror surface.

A navigation device for the acetabular cup, which has the aforementioned structure, operates in the following manner.

The first reference mechanism is the same as in the aforementioned embodiments. When the first reference mechanism is aligned on the pelvis, the rod-shaped reference mechanism indicates a direction perpendicular to a specific reference plane. When the second reference mechanism is aligned with respect to the first reference mechanism, the reference mechanism having the mirror surface is aligned such that the laser beam emitted from the laser level tool of the first reference mechanism is reflected along an incident path of the laser beam, and the rod-shaped reference mechanism of the second reference mechanism is parallel to the rod-shaped reference mechanism of the first reference mechanism.

While the present invention has been particularly shown and described with reference to exemplary embodiments thereof, it will be understood by those skilled in the art that various changes in form and details may be made therein without departing from the spirit and scope of the invention as defined by the appended claims.

For example, the thickness of a subcutaneous layer in each body part in contact with probes is not uniform, and thus, a plane formed by three probes attached thereto may be slightly deviated from a pelvic plane. To prevent this, a sensor, that is, a caliper, for measuring the thickness of the subcutaneous layer may be attached to the probes' respective ends in contact with the human body. The caliper attached to the probes measures the thickness of the subcutaneous layer of the body part in contact with the caliper, and compensates for an angle of a plane detected by taking the measured thickness of the subcutaneous layer into account, thereby tracing a plane which is the most similar to an actual pelvic plane. The thickness of the subcutaneous layer is different depending on the level of obesity, but in general, the thickness of the subcutaneous layer is greater in the symphysis pubis than in the left and right anterior superior iliac spines. Therefore, the sensor may be attached to the probe in contact with the symphysis pubis only. The caliper may use a skin-fold method in which skin is folded in the thickness measuring, and an ultrasonic wave method using an ultrasonic wave. Preferably, the caliper uses the ultrasonic wave method in the present invention.

### INDUSTRIAL APPLICABILITY

According to the present invention, an insertion orientation of an acetabular cup can be guided by using a reference mechanism having a simple structure, without the use of optical and electrical devices which are complex and expensive.

In addition, the acetabular cup can be accurately guided regardless of changes in the patient's pelvic position during surgery, because a plane used in the insertion of the acetabular cup can be indicated continuously.

1. A navigation system for an acetabular cup, which guides an insertion orientation of the acetabular cup inserted into a pelvis during a total hip replacement surgery, and uses reference mechanisms, the navigation system comprising:
a pelvis position tracer which includes probes in contact with three particular points of the pelvis placed on an anterior pelvic plane and a first reference mechanism disposed to indicate a specific reference plane when the probes come in contact with the particular points; and
a pelvis position indicator which is fixed to the pelvis, and includes a second reference mechanism that is adjustable to indicate a plane parallel to the specific reference plane indicated by the first reference mechanism, or to indicate a plane perpendicular thereto, or to indicate the both planes.

2. The navigation system according to claim 1, wherein the probes of the pelvis position tracer comprise a first probe in contact with a symphysis pubis, a second probe in contact with a left anterior superior iliac spine, and a third probe in contact with a right anterior superior iliac spine.

3. The navigation system according to claim 2, wherein the first probe has a curve portion at its end in contact with the symphysis pubis, thereby forming the L-shape.

4. The navigation system according to claim 1, wherein the specific reference plane indicated by the first reference mechanism is an anterior pelvic plane and/or a sagittal plane.

5. The navigation system according to claim 1, wherein the pelvis position indicator further comprises one or more pins fixed to the pelvis, a base supported by the pins, a ball joint extended from the base, and a fixed block which is connected to the ball joint and at which the second reference mechanism is disposed.

6. The navigation system according to claim 5, wherein the fixed block is made of a shape memory alloy, is separated from the ball joint after being transformed in a specific temperature range higher than a room temperature, and is fixed firmly to the ball joint in the room temperature.

7. The navigation system according to claim 5, wherein the fixed block comprises two blocks connected with each other by means of a screw.

8. The navigation system according to claim 1, wherein the first and second reference mechanisms are flat polygonal plane-shaped.

9. The navigation system according to claim 1, wherein the first and second reference mechanisms are one or more rod-shaped members.

10. The navigation system according to claim 1, wherein the pelvis position indicator further comprises a third reference mechanism indicating a plane tilted at a specific angle with respect to a plane indicated by the second reference mechanism.

11. The navigation system according to claim 10, wherein the plane indicated by the third reference mechanism of the pelvis position indicator is perpendicular to the plane indicated by the second reference mechanism.

12. The navigation system according to claim 1, wherein the first reference mechanism is a laser level tool which emits a laser beam indicating the specific reference plane, and the second reference mechanism is a flat polygonal plane-shaped member having a plurality of through-holes through which the laser beam emitted from the laser level tool passes.

13. The navigation system according to claim 1, wherein the first reference mechanism comprises a laser indicator emitting a laser beam indicating the specific reference plane and a rod-shaped reference mechanism indicating a direction perpendicular to the specific reference plane, and the second reference mechanism comprises a reference mechanism having a mirror surface to reflect a laser beam emitted from the laser indicator and a rod-shaped reference mechanism fixed parallel to the mirror surface of the reference mechanism having the mirror surface, and
when the second reference mechanism is aligned with respect to the first reference mechanism, the reference mechanism having the mirror surface is aligned such that the laser beam emitted from the laser level tool of the first reference mechanism is reflected along an incident path of the laser beam, and the rod-shaped reference mechanism of the second reference mechanism is parallel to the rod-shaped reference mechanism of the first reference mechanism.

14. The navigation system according to claim 1, further comprising: a caliper which is attached to at least one of the probes' respective ends in contact with the human body, and measures the thickness of the subcutaneous layer; and a compensating tool compensating for an angle of a plane based on the result obtained by measuring the thickness of the subcutaneous layer.

15. The navigation system according to claim 14, wherein the caliper measures the thickness of the subcutaneous layer using an ultrasonic wave method.

16. The navigation system according to claim 14, wherein the caliper is provided at a probe in contact with the symphysis pubis.

17. A method of guiding an insertion orientation of an acetabular cup inserted into a pelvis during a total hip replacement surgery by using a navigation system for the acetabular cup, where the navigation system uses the reference mechanisms of claim 1, the method comprising:
(a) fixing the pelvis position indicator to the pelvis;
(b) allowing the first reference mechanism to indicate a specific reference plane by arranging the pelvis position tracer so that the probes of the pelvis position tracer come in contact with the three particular points;
(c) fixing the second reference mechanism of the pelvis position indicator after adjusting the second reference mechanism to indicate a plane parallel to the specific reference plane indicated by the first reference mechanism of the pelvis position tracer and/or a plane perpendicular the plane parallel to the specific reference plane;
(d) removing the pelvis position tracer arranged on the pelvis;
(e) separating the pelvis position indicator from the pelvis;
(f) disposing the pelvis to a desirable position;
(g) fixing again the pelvis position indicator to the position where the pelvis position indicator is fixed in the (a); and
(h) navigating the acetabular cup with reference to the plane indicated by the second reference mechanism of the pelvis position indicator.

18. The method according to claim 17, wherein the specific reference plane is an anterior pelvic plane and/or a sagittal plane.

19. The method according to claim 17, wherein the (b) comprises:
(b-1) allowing the first reference mechanism to indicate a first reference plane by arranging the pelvis position tracer so that the probes of the pelvis position tracer come in contact with the three particular points;
(b-2) measuring the thickness of the subcutaneous layer at a body part in contact with at least one of the probes; and
(b-3) tracing the specific reference plane by compensating for the first reference plane measured in the (b-1) based on the thickness of the subcutaneous layer measured in the (b-2)
